(19) United States
(12) Patent Application Publication
(10) Pub. No.: US 2015/0045258 A1
(43) Pub. Date: Feb. 12, 2015

(54) CASCADED ADDITION OF TARGET SPECIFIC UNIVERSAL ADAPTERS TO NUCLEIC ACIDS

(71) Applicant: GnuBio, Inc., Cambridge, MA (US)

(72) Inventors: Tal Raz, Brookline, MA (US); Haifa Ghandour, Watertown, MA (US); Pascaline Mary, Cambridge, MA (US)

(21) Appl. No.: 14/377,964

(22) PCT Filed: Feb. 8, 2013
(86) PCT No.: PCT/US13/25274

§ 371 Date: Aug. 11, 2014

Related U.S. Application Data
(60) Provisional application No. 61/598,442, filed on Feb. 14, 2012.

(51) Int. Cl. C12N 15/10; C12Q 1/68

(52) U.S. Cl. CPC C12N 15/1065; C12Q 1/6853
USPC 506/26; 435/6.12

(57) ABSTRACT

The present invention generally pertains to methods for the addition of one or more nucleic acid adaptors to a PCR product. The present invention generally relates to a method for the cascaded, highly specific addition of universal nucleic acid adapters to one or more target nucleic acids in a single PCR amplification reaction, as well as a kit encompassing the same.

### FEDERAL FUNDING LEGEND

This invention was supported, in part, by NHGRI grant number: 1R43HG005144-01. The federal government may have certain rights to this invention.

### RELATED APPLICATIONS AND INCORPORATION BY REFERENCE

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention is in the technical field of biotechnology. More particularly, the present invention is in the technical field of molecular biology.

### BACKGROUND OF THE INVENTION

The incorporation of universal nucleic acid adapters is often used in nucleic acid manipulations as a means to incorporate the nucleic acid into a functional system. For example, adapters may be added to incorporate a fluorescent molecule into a sample of interest, to add a nucleic acid sequence that can later be utilized as part of a biochemical assay, or to assign a unique identifier to a sample before mixing it with other uniquely labeled samples (as has been done, for example, in sequencing mixed nucleic acid samples from multiple human or animal subjects or patients).

One of the limitations in adding adapters to nucleic acids is that it requires the assay design to be specifically tailored for a single application. For example, PCR primers are often designed with an added adapter sequence so that they can be used for specific interrogation assays. However, a new design of PCR primers is required for additional assays. Primer redesign is both costly and time consuming particularly since adapter sequences often require expensive nucleic acid modification. Therefore, the addition of such modifications to each PCR primer set designed becomes prohibitive. Accordingly, there is a need for an affordable and efficient method for the addition of nucleic acid adapters to a PCR product in a single reaction.

The following invention provides a method for the cascaded addition of nucleic acid adapters to a PCR product in a single reaction. Moreover, the invention provides a method wherein tailored adaptors can be added to essentially any PCR product in a single reaction. Furthermore, the invention facilitates the use of a single adapter that is universally designed to complement a multitude of target nucleic acids.

Citation or identification of any document in this application is not an admission that such document is available as prior art to the present invention.

### SUMMARY OF THE INVENTION

The present invention generally pertains to methods for the addition of nucleic acid adaptors to a PCR product. One embodiment of the method according to the present invention provides for the cascaded addition of nucleic acid adapters to a PCR product in a single reaction. The adapters may be tailored such that they can be added to essentially any PCR product in a single reaction. One aspect of the method facilitates the use of a single adapter that may be universally designed to complement a multitude of target nucleic acids of interest. Another aspect of the method provides for the cascaded, highly specific addition of universal nucleic acid adapters to a target nucleic acid in a single PCR amplification reaction.

The present invention also pertains to a kit comprising the reagents and instructions for performing methods for the addition of nucleic acid adaptors to a PCR product. One embodiment of the kit according to the present invention provides the reagents for the cascaded addition of nucleic acid adapters to a PCR product in a single reaction. The adapters may be tailored such that they can be added to essentially any PCR product in a single reaction. One aspect of the kit provides the reagents and instructions to facilitate the use of a single adapter that may be universally designed to complement a multitude of target nucleic acids of interest. Another aspect of the kit provides the reagents and instructions for the cascaded, highly specific addition of universal nucleic acid adapters to a target nucleic acid in a single PCR amplification reaction.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWING

The following detailed description, given by way of example, but not intended to limit the invention solely to the specific embodiments described, may best be understood in conjunction with the accompanying drawings.

FIG. 1 is an illustration of the design and orientation of a target specific (“TS”) primer and a universal (“U”) primer.

FIG. 2 is an illustration of one embodiment of the method according to the present invention, comprising a four primer cascade.

### DETAILED DESCRIPTION OF THE INVENTION

The present invention generally pertains to methods for the addition of nucleic acid adaptors to a PCR product. One embodiment of the method according to the present invention provides for the cascaded addition of nucleic acid adapters to a PCR product in a single reaction. The adapters are tailored such that they can be added to essentially any PCR product in a single reaction. One aspect of the method facilitates the use of a single adapter that is universally designed to complement a multitude of target nucleic acids of interest. Another aspect of the method provides for the cascaded, highly specific addition of multiple universal nucleic acid adapters to a target nucleic acid in a single PCR amplification reaction.

The methods of the invention may further comprise the use of a polymerase enzyme. This may be any enzyme with strand-displacement capacity. Examples of commercially available polymerase enzymes include, but are not limited to: Klenow fragment (New England Biolabs® Inc.), Taq DNA polymerase (QIAGEN), 9° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.).

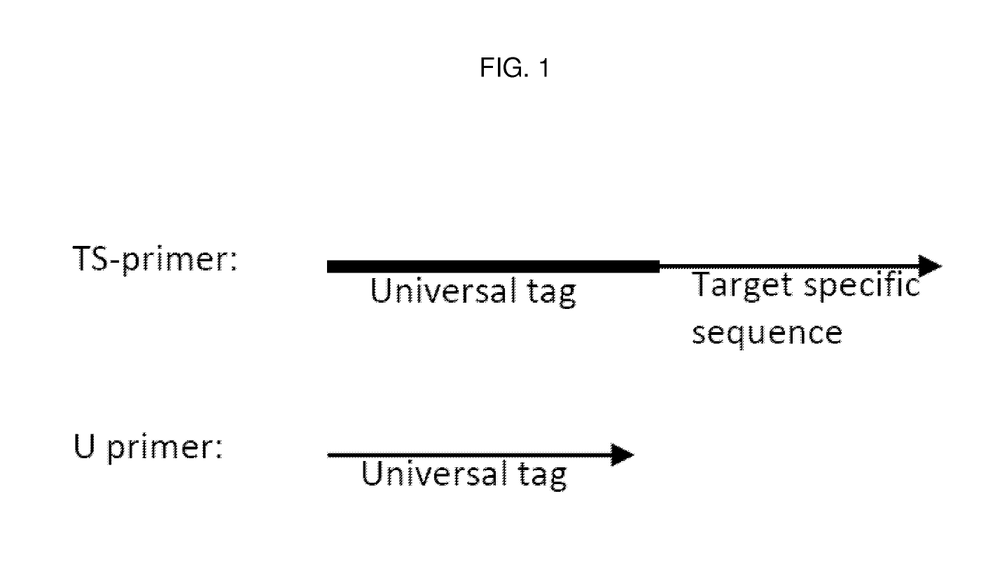

In one aspect of this embodiment, the method provides for the addition of universal adapter sequences onto a target nucleic acid in a PCR amplification reaction. The method comprises at least two target specific (“TS”) primers and at least one universal (“U”) primer, referred to herein as “TS primer(s)” or “TS primer set” and “U primer” or “U primer set”, respectively. The TS primers are designed in view of the target nucleic acid sequence of interest. The U primers have a universal design such that they are compatible with virtually any TS primer set. As illustrated in FIG. 1, each TS primer comprises a 3′ region specific to the target nucleic acid sequence of interest and a 5′ region comprising a universal nucleic acid sequence. The U primer comprises an adapter sequence comprising a nucleic acid sequence that is added to the target nucleic acid so that the target nucleic acid can be interrogated by an assay system. Assay systems may include but are not limited to, an instrument, a chemical assay or other interrogation technology. For example, adapter sequences may be used to anchor DNA to a solid surface as is done when using DNA sequencing technology. In another example, adapter sequences are used to facilitate binding of DNA to streptavidin beads. In yet another example, adapter sequences are used to add fluorophores or quenchers to molecules for optical detection assays. The U primer further comprises a universal tag sequence substantially matching the universal nucleic acid sequence of each TS primer such that this U primer and TS primer are referred to herein as a “primer pair”. In some aspects, the adapter sequence and the universal tag sequence may be one and the same or overlapping in some respects.

The method of this embodiment comprises at least two primer sets, wherein each primer set comprises at least one primer but may comprise any number of primers including, but not limited to, two or more primers. Additionally, the amplification reaction may comprise any combination of numbers and types of primers and any combination of numbers and types of primer sets. In one aspect, the method may comprise three primers, referred to herein as a “three primer cascade”. In one example of this aspect, two TS primers and one U primer may be utilized. In yet another aspect, the method may comprise four primers, referred to herein as a “four primer cascade”. In one example of this aspect, the four primers include but are not limited to two TS primers and two U primers. In still other aspects, the method may comprise more than four primers.

In another aspect of this embodiment, the method provides for the addition of universal adapter sequences onto a target nucleic acid in a multiplexed PCR amplification reaction. In this aspect, one or more primers against more than one nucleic acid sequence may be used in a single PCR reaction. For example, one or more TS primers against one or more nucleic acid sequences, wherein the one or more nucleic acid sequences are associated with one or more gene sequences, may be used in a single reaction together with one U primer or, alternatively, with more than one U primer.

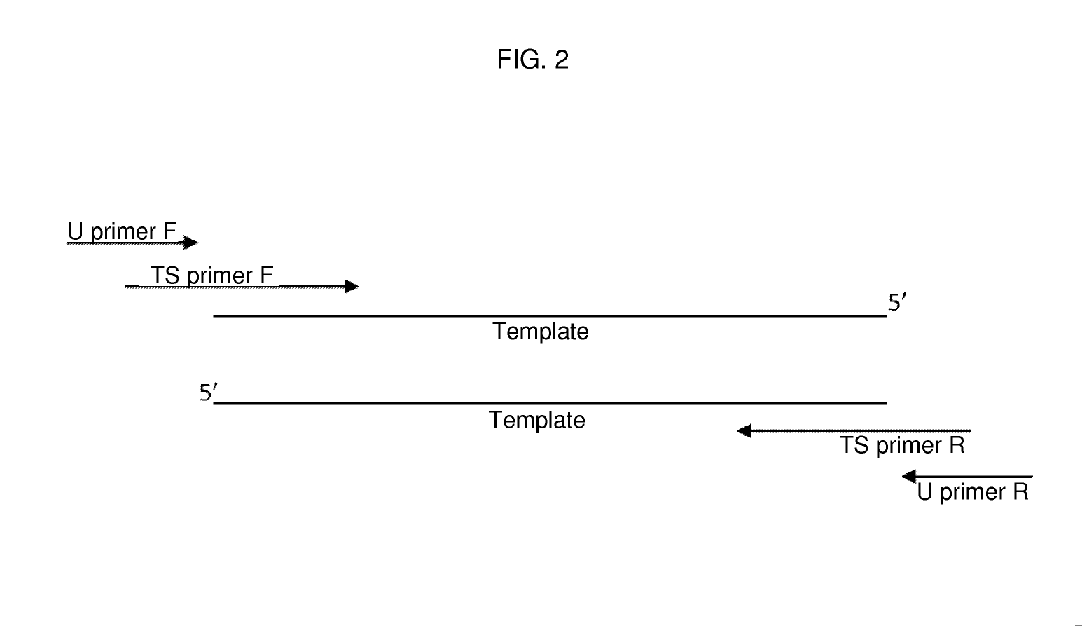

Referring now to FIG. 2, which illustrates one embodiment of the method according to the present invention, comprising a four primer cascade. In this embodiment, the four primer cascade comprises a TS primer set and a U primer set. The TS primer set comprises TS primer F and TS primer R (collectively, the “TS primers”). The U primer set comprise U primer F and U primer R (collectively, the “U primers”). The primers in the four primer cascade associate with each other to form two primer pairs. The first primer pair in this embodiment comprises TS primer F and U primer F, wherein TS primer F comprises a 3′ region specific to the target nucleic acid sequence of interest and a 5′ region comprising a universal nucleic acid sequence, and wherein U primer F comprises a universal tag substantially matching the universal nucleic acid sequence of the TS primer F. The second primer pair in this embodiment comprises TS primer R and U primer R, wherein TS primer R comprises a 3′ region specific to the target nucleic acid sequence of interest and a 5′ region comprising a universal nucleic acid sequence, and wherein U primer R comprises a universal tag substantially matching the universal nucleic acid sequence of the TS primer R.

The U primers further comprise any additional sequence or modification required for the assay of interest, including but not limited to, a “fluorescent label”, “fluorophore” or “fluorescent dye”, each of which is used herein to collectively include a fluorescent molecule, a fluorescent semiconductor nanoparticle (referred to as a “quantum dot”), or a chelated lanthanide or lanthanoid, having the ability to absorb energy from light of a specific wavelength, and then emit this energy as fluorescence in another specific wavelength characteristic for the particular molecule or quantum dot. In this manner, the fluorophore will facilitate the final assay readout indicating the presence or absence of a particular target of interest in the sample.

The particular fluorophore employed is not critical to the present invention. Fluorophores are known in the art and are described, for example, by Marras, “Selection of Fluorophore and Quencher Pairs for Fluorescent Nucleic Acid Hybridization Probes”, In: V. Didenko, ed. 2006. Fluorescent Energy Transfer Nucleic Acid Probes: Designs and Protocols (Methods in Molecular Biology, vol. 335). New Jersey: Humana Press Inc., pp.3-16. Examples of fluorophores that can be employed in the present invention include, but are not limited to, those described by Marras 2006 and further described herein below. The particular location of the fluorophore in relation to the detector is not critical to the present invention. The fluorophore can be attached anywhere along the detector, including the 5′ end, the 3′ end or anywhere internally along the detector.

Examples of the specific fluorophores that may be employed in the present invention include, but are not limited to fluorescein and derivatives thereof (e.g., fluorescein isothianate (FITC), carboxyfluorescein (FAM), tetrachlorofluorescein (TET), 2′,7′-difluorofluorescein (Oregon Green® 488), Oregon Green® 514 carboxylic acid, and a fluorescein with chloro and methoxy substituents (JOE and 6-JOE)); rhodamine derivatives (e.g., tetramethyl rhodamine (TAMRA), tetramethyl rhodamine iso-thiocyanate (TRITC), tetramethylrhodamine (TMR), carboxy-X-rhodamine (ROX), Texas Red (a mixture of isomeric sulfonyl chlorides and sulforhodamine; Invitrogen™) and Texas Red-X (Texas Red succinimidyl ester, which contains an additional seven-atom aminohexanoyl spacer (“X”) between the fluorophore and its reactive group; Invitrogen™), and Rhodamine X); cyanine (Cy) dyes (e.g., Cy3, Cy5 and Cy5.5) and cyanine derivatives (e.g., indocarbocyanine (Quasar® 570, Quasar® 670 and Quasar® 705), Oregon Green® isothiocyanate, and eosin isothiocyanate (EITC)); N-hydroxysuccinimidyl 1-pyrenebutyrate (PYB); N-hydroxysuccinimidyl 1-pyrenesulfonate (PYS); (5-(2′-aminoethyl)aminonaphthalene (EDANS); CAL Fluor® Gold 540, CAL Fluor® Orange 560, Fluor® Red 590, CAL Fluor® Red 610, and CAL Fluor® Red 635 (proprietary fluorophores available from Biosearch Technologies, Inc.); VIC®; HEX® (a 6-isomer phosphoramidite); and NED®.

The U primers may further comprise deoxyuridines, additional nucleic acid sequences, and so forth. Furthermore, the TS primers are designed to have a relatively higher melting temperature (Tm) than that of the U primers. This Tm difference serves to ensure the specificity of primers in the PCR reaction of the method.

The “PCR product” as used in the methods of this invention is the amplification product of a target nucleic acid. The “target nucleic acid” as used in the methods of this invention is a nucleic acid sample obtained from a human, animal, plant or any other organism or microorganism, and includes, but is not limited to, genomic DNA, mitochondrial DNA, cDNA, and others. The target nucleic acid may be double stranded or single stranded. In one embodiment, a double stranded target nucleic acid is first converted to a single stranded target nucleic acid. In one aspect of this embodiment, the PCR product is subsequently converted to single stranded form.

In one aspect of this embodiment, the PCR product comprises the amplified product of a single nucleic acid target. In another aspect of this embodiment, the PCR product comprises the amplified product of a multitude of nucleic acid targets. In yet another aspect of this embodiment, the PCR product comprises the amplified product of a particular nucleic acid sequence variance within the target nucleic acid. A “variance” is a difference in the nucleotide sequence among related polynucleotides. The difference may be the deletion of one or more nucleotides from the sequence of one polynucleotide compared to the sequence of a related polynucleotide, the addition of one or more nucleotides or the substitution of one nucleotide for another. The terms “mutation,” “polymorphism” and “variance” are used interchangeably herein. As used herein, the term “variance” in the singular is to be construed to include multiple variances, i.e., two or more nucleotide additions, deletions and/or substitutions in the same polynucleotide. A “point mutation” refers to a single substitution of one nucleotide for another.

For example, the PCR product may comprise the amplified product of a single nucleotide polymorphism. A “single nucleotide polymorphism” or “SNP” refers to a variation in the nucleotide sequence of a polynucleotide that differs from another polynucleotide by a single nucleotide difference. A SNP included, for example and without limitation, exchanging one A for one C, G or T, or one C for one G, T or C and so on, in the entire sequence of polynucleotide. Additionally, it is possible to have more than one SNP in a particular nucleic acid sequence. For example, at one position in a nucleic acid sequence, a G may be exchanged for an A, at another position a C may be exchanged for a T and so on. In another example, the PCR product may comprise the amplified product of a single nucleotide mutation.

In yet another example, the PCR product may comprise the amplified product of more than one nucleotide mutation. In another embodiment, the PCR product comprises the amplified product of a plurality of nucleic acid targets (e.g., two or more genomic regions). In one aspect of this embodiment, the PCR product comprises the amplified product of a short nucleic acid sequence, including but not limited to, about six to about eight nucleotides. In another aspect of this embodiment, the PCR product comprises the amplified product of an entire DNA sequence. Accordingly, the PCR product has no specific length limitation.

The results of the methods of this invention, referred to herein as “data”, associated with a particular target nucleic acid sequence or PCR product may then be kept in an accessible database, and may or may not be associated with other data from that particular human or animal associated with the target nucleic acid sequence or with data from other humans or animals. Data obtained may be stored in a database that can be integrated or associated with and/or cross-matched to other databases.

The methods and kits of this invention may further be associated with a network interface. The term “network interface” is defined herein to include any person or computer system capable of accessing data, depositing data, combining data, analyzing data, searching data, transmitting data or storing data. The term is broadly defined to be a person analyzing the data, the electronic hardware and software systems used in the analysis, the databases storing the data analysis, and any storage media capable of storing the data. Non-limiting examples of network interfaces include people, automated laboratory equipment, computers and computer networks, data storage devices such as, but not limited to, disks, hard drives or memory chips.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

In one example of the method of the present invention, a PCR reaction is performed using standard PCR reaction components (target nucleic acid, dNTPs, reaction buffer, DNA polymerase, and magnesium chloride (MgCl)), wherein the reaction components are mixed with a relatively low concentration of TS primers and a relatively high concentration of U primers. The TS primers may be added at a final concentration of 10 nM and the U primers may be added at a final concentration of 200 nM. The concentration difference between the primer sets ensures that the final PCR product will contain the U primer adapters. The PCR reaction is amplified by thermocycling as follows:
1. A number of PCR cycles (e.g., 15 cycles) are run at a high annealing temperature that is close to the Tm of the TS-primers and well above the Tm of the U primers.
2. The remaining PCR cycles (e.g., 25 cycles) are done with a lower annealing temperature that is near the Tm of the U primers.

The use of different annealing temperatures during the PCR amplification reaction above ensures the specificity of the reaction, such that target specific amplification occurs at the early cycling steps and depletes the TS primers. The U primers then readily amplify the enriched specific target such that the final PCR product contains the U primer adapters. The approach of using different annealing temperatures resolves problems of nonspecific amplification often seen in multiplexed PCR reactions.

### Example 2

In another example of the method of this invention, a PCR reaction is performed using multiple TS primers to simultaneously amplify any number of nucleic acid targets. Standard PCR reaction components are included (target nucleic acid, dNTPs, reaction buffer, DNA polymerase, and magnesium chloride (MgCl)), wherein the reaction components are mixed with a relatively low concentration of TS primers, e.g., 10 nM, and a relatively high concentration of U primers, e.g., 200 nM. Each TS primer has a relatively high Tm (e.g., 67° C.) and each U primer has a relatively low Tm (e.g., 45° C.). The PCR reaction is amplified by thermocycling as described for Example 1.

### Example 3

In yet another example of the method of this invention, a PCR reaction is performed using multiple TS primers and multiple U primers to amplify any number of nucleic acid targets simultaneously. Each TS primer is designed to match a different U primer. Accordingly, multiple U primers are included such that each U primer matches a different TS primer set. PCR amplification will result in a number of different amplicons each tagged with a unique U primer adapter.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method for the addition of nucleic acid adapters to a PCR product comprising:
a) performing a PCR reaction comprising a mixture of PCR reagents, a target nucleic acid, at least one target specific (“TS”) primer set, and at least one universal (“U”) primer set;
b) wherein the at least one TS primer set comprises at least two TS primers and the at least one U primer set comprises at least one U primer;
c) wherein the at least one TS primer set and the at least one U primer set together comprise a primer pair; and
d) amplifying the one or more target nucleic acid by thermocycling the above mixture, wherein the resulting PCR product contains nucleic acid adapters.

2. A method according to claim 1, wherein each TS primer comprises a 3′ region specific to the target nucleic acid.

3. A method according to claim 2, wherein each TS primer further comprises a 5′ region comprising a universal nucleic acid sequence.

4. A method according to claim 3, wherein each U primer comprises a universal tag sequence substantially matching at least part of the universal nucleic acid sequence of each TS primer in the at least one TS primer set in the primer pair.

5. A method according to claim 4, wherein each U primer comprises an adapter sequence.

6. A method according to claim 5, wherein the concentration of each U primer is greater than the concentration of each TS primer.

7. A method according to claim 5, wherein the melting temperature of each U primer is lower than the melting temperature of each TS primer.

8. A method according to claim 7, wherein two or more PCR cycles are performed with an annealing temperature substantially similar to the melting temperature of the TS primer, followed by the performance of two or more PCR cycles with annealing temperature substantially similar to the melting temperature of the U primer.

9. A method according to claim 5, wherein the addition of nucleic acid adapters to a PCR product is performed in a cascaded manner.

10. A method according to claim 5, wherein the addition of nucleic acid adapters to a PCR product is performed in a single reaction.

11. A method according to claim 5, wherein each U primer further comprises one or more additional sequences or modifications selected from fluorescent molecules, quantum dots, deoxyuridines, and/or additional nucleic acid sequences.

12. A method according to claim 1, comprising two or more TS primer sets and two or more nucleic acid targets, wherein the method provides for simultaneous amplification of two or more nucleic acid targets.

13. A method according to claim 12, comprising two or more U primers, wherein each TS primer is designed to match a different U primer.

14. A kit for performing the method according to claim 1.
